(19) United States
(12) Patent Application Publication
(10) Pub. No.: US 2023/0280826 A1
(43) Pub. Date: Sep. 7, 2023

(54) MAGNETIC BRAIN COMPUTER INTERFACE INJECTOR AND METHODS OF USING SAME

(71) Applicant: Austin J. Georgiades, Marlborough, CT (US)

(72) Inventor: Austin J. Georgiades, Marlborough, CT (US)

(21) Appl. No.: 17/687,783

(22) Filed: Mar. 7, 2022

(51) Int. Cl. G06F 3/01; A61B 17/00

(52) U.S. Cl. CPC G06F 3/015; A61B 17/00234; A61B 2560/063

(57) ABSTRACT

In some examples, a computer-brain interface injector, includes a hollow tube having a longitudinal axis and configured to receive at least a portion of an interface, and at least two arms capable or radial extension in a direction normal to the longitudinal axis of the hollow tube.

### CROSS-REFERENCE TO RELATED APPLICATIONS

This application claims priority to U.S. Provisional Ser. No. 63/236,611, filed on Aug. 24, 2021, the contents of which is hereby incorporated by reference in its entirety as if fully set forth herein.

### FIELD OF THE DISCLOSURE

The present disclosure relates to devices and methods for creating a brain-computer interface. More particularly, the present disclosure relates to injectors and methods of delivering brain-computer interface.

### BACKGROUND OF THE DISCLOSURE

Brain-computer interfaces (BCI) are systems that provide communications between biological neural networks and some sort of electronic, computational device. BCIs can be used, for example, by individuals to control an external device such as a wheelchair using neural activity which is read from their brain. A major goal of brain-computer interfaces (BCIs) is to decode intent from the brain activity of an individual, and respond to said intent in a desired way. One example of the promise of BCIs relates to aiding people with severe motor impairments. However, conventional devices and methods are unnecessarily invasive. Additionally, there are no simple ways of delivering and implanting these interfaces in an atraumatic manner.

### BRIEF DESCRIPTION OF THE DISCLOSURE

Various embodiments of the presently disclosed devices and methods are described herein with reference to the drawings, wherein:

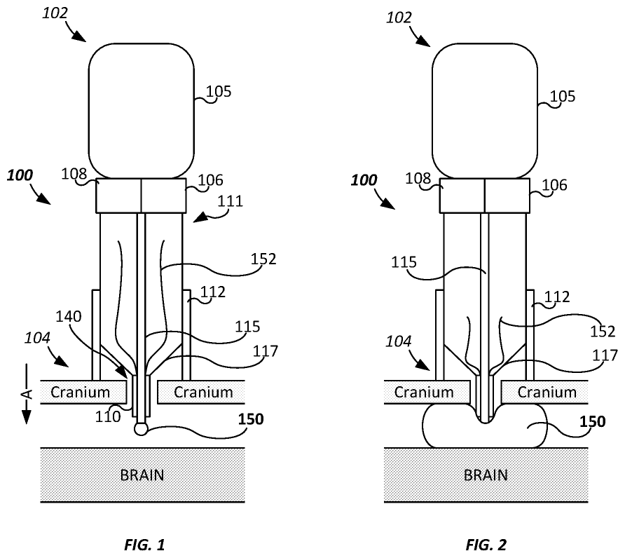

FIGS. 1-3 are simplified schematic representations of an injector system for a magnetic brain computer interface surface membrane at various steps during the procedure;

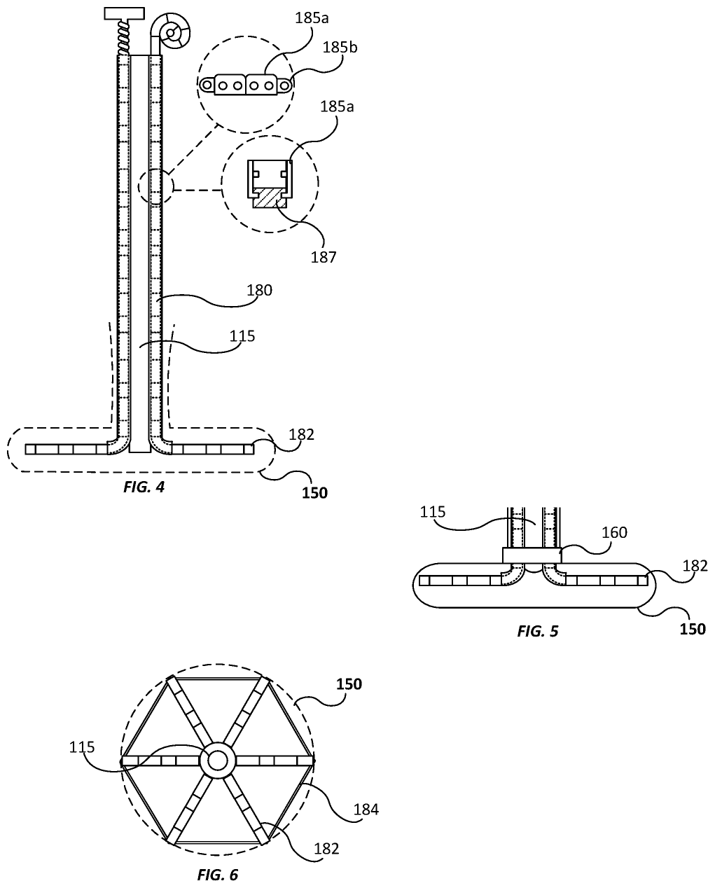

FIG. 4 is a simplified schematic representation showing injector system including radial arms;

FIG. 5 is a simplified schematic representation showing a brain interface interacting with the radial arm injector system;

FIG. 6 is a simplified schematic top view showing a brain interface interacting with the radial arm injector system; and

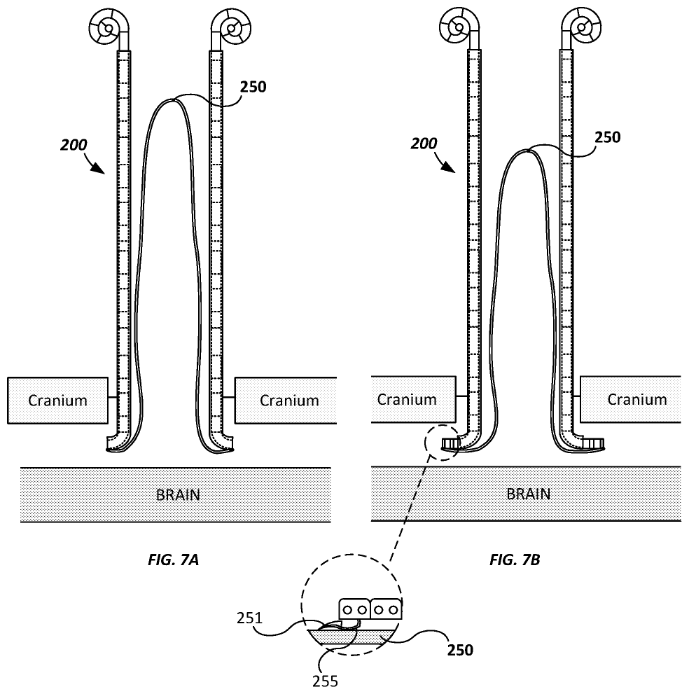

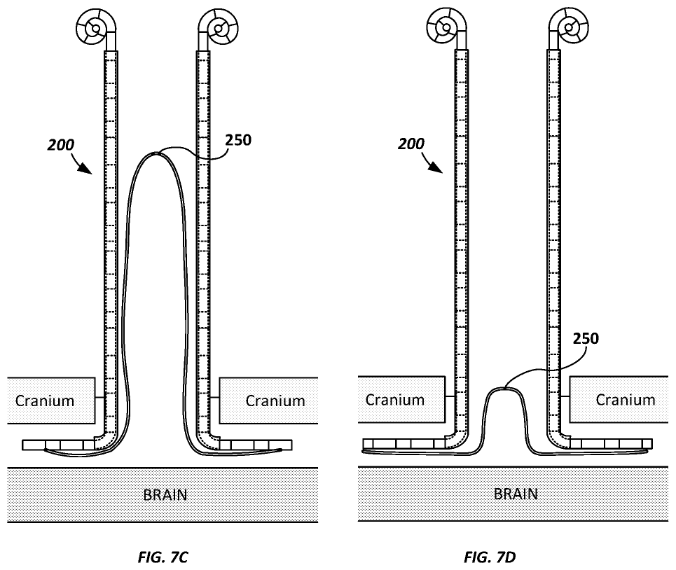

FIGS. 7A-D are schematic representations showing a non-cavernous brain interface interacting with the non-cavernous interface injection system.

Various embodiments of the present invention. will now be described with reference to the appended drawings. It is to be appreciated that these drawings depict only some embodiments of the invention and are therefore not to be considered limiting of its scope.

### DETAILED DESCRIPTION

Despite the various improvements that have been made to brain-computer interfaces (BCIs), conventional devices and their methods of implantation suffer from some shortcomings. Devices which measure and stimulate from outside the skull are significantly limited. in. resolution whereas current invasive BCIs require extensive surgical intervention to implant and are therefore limited in scope.

There therefore is a need for further improvements to the devices, systems, and methods of delivering and implanting brain-computer interfaces. Among other advantages, the present disclosure may address one or more of these needs.

As used herein, the term “proximal,” when used in connection with a component of a brain-computer interface, refers to the side of the component closest to or in contact with the brain when the brain-computer interface is implanted in a patient, whereas the term “distal,” when used in connection with a component of a brain-computer interface, refers to the end of the component farthest from the brain when the assembly is inserted in a patient. More generally, “proximal” may refer to component side closest to implantation region whereas “distal” refers to component side furthest from implantation region.

In some embodiments, a mechanism is described to facilitate the surgical implantation of a magnetic brain computer interface surface membrane or interface 150. Details of similar interfaces are more fully described in U.S. Ser. No. 17/390,541, entitled “MAGNETIC BRAIN COMPUTER INTERFACE SURFACE MEMBRANE AND METHODS OF USING SAME,” filed Jul. 30, 2021, and U.S. Provisional Ser. No. 63/069,046, entitled “MAGNETIC BRAIN COMPUTER INTERFACE SURFACE MEMBRANE,” filed Aug. 22, 2022, the disclosures of which are hereby incorporated in their entirety as if fully set forth herein.

With reference to FIG. 1, generally, an injector 100 extends between a distal end 102 and a proximal end 104, and may include two distinct subsystems, the pump system and the injector support structure 111, which are designed to interact or fit together to produce a compound structure which can inflate an interface 150 to fill the space between the action potential producing tissue (APT) and the tissue which surrounds it, while expanding to cover a broad surface of the APT. In some examples, the pump system may include a reversible electric pump 108 driven by a power source 106, a reservoir 105 for fluid storage, and a tubular nozzle 115 through which fluid is pumped. The injector 100 may further include a generally tubular tension encasement 110, a surface brace 112, and an interiorly-disposed guiding surface 117.

In FIG. 1, a fluid reservoir 105 is shown connected to a fluid pump 108 and a power source 106. The power source 106 drives the pump 108 to push fluid from the reservoir 105 into the interior of the interface 150 at the end of the nozzle 116. Flow can also be reversed in case of emergency to provide suction. The nozzle 115, the power source 106, the pump 108 and the reservoir 105 may form one contiguous primary unit of injector 100. The lower sides of the pump and power source are made to effectively link with the injector support structure 111, which, along with surface braces 112 and tensioning encasement 110, forms a secondary contiguous unit that can be attached to and unattached from the primary unit. Notice how the tensioning encasement 110 encircles or otherwise tightly envelopes nozzle periphery distal guiding surface 117 to press the interface 150 against nozzle 115. Prior to this stage of injection, the two units are apart. An interface, which resembles a hollow pancake with a tuft of excess membrane surrounding an opening in the top, (centralized on one of the broadest, flat sides, for instance) may be placed over the nozzle and stretched over the nozzle until the tip of the nozzle is in contact with the point on the interface that is antipolar to the opening and the interface spans the greatest nozzle length. At this point, the two units may be attached, joined or otherwise coupled or made in mechanical engagement with one another, with the tensioning encasement opened to allow the interface-wrapped nozzle through and then allowed to clamp down once in position. Surface-braces are then adjusted to position the tip of the nozzle at the correct depth for inflation.

In some examples, it may be desirable to place an interface 150 against the surface of the brain so that it covers as much surface area as possible. Typically, the interface will be placed between the brain (or APT) and the cranial meningeal surface in the subarachnoid space. The interface 150 may be inflated within the subarachnoid space using injector 100, sliding off the nozzle 115 of the syringe-pump as fluid is forced into it and forming a gradually increasing bubble within the space between the brain and the arachnoid mater.

An outer receiving portion called the tensioning encasement 110 may pinch the interface 150 against nozzle 115 slightly distal respectively relative to the nozzle's proximal opening to prevent pumped fluid from escaping the bubble, thus forcing the interface to inflate into a balloon-like shape much like water balloons when inflated and filled with water. As the rest of the interface 150 (e.g., the portions that make up free tail 152 of the interface) is not yet part of the inflation bubble slides through the tensioning encasement, the bubble may inflate to occupy the volume between the brain and cranial meningeal surface within subarachnoid space, thus enabling the interface to contact a large surface area of brain using a minimally-invasive surgical portal 140.

Prior to loading interface 150 for insertion, the outside and inside of the interface 150 may be first lubricated with biocompatible lubricant to minimize friction against the tensioning encasement 110 and nozzle 115 respectively. Outside lubrication may also help the inflating interface 150 to slide across the arachnoid mater once off the nozzle and within the subarachnoid space, thus facilitating its internal inflation over the brain. The nozzle 115 of the pump system may be placed through an opening at the top of an interface and the interface may then be pulled over the nozzle until it is stretched over a majority or the full length of the nozzle to greatest extent which does not cause nozzle to pierce the interface and the region of interface opposite this opening when hypothetically inflated to fill desired body space and resting after implantation is in contact with the opening of the nozzle.

Then, the pump unit support structure 111 and tensioning encasement 110 may be placed around the tip of the nozzle and locked into place so that it does not move when fluid is pumped into the bubble formed by the tensioning encasement, which causes the interface 150 to slide off the nozzle 115 as bubble expands requiring more interface surface area be drawn through tensioning encasement to form the larger surface area of expanding bubble. If the tensioning encasement were to move, it could slip off the tip of the nozzle, being dragged by the interface 150 sliding through it, and break the seal of the bubble after sliding off nozzle, thus compromising inflation. Optionally, a circular tension-ring 160, best seen in FIG. 5, at the base of the tension encasement is loosened to allow the interface-wrapped nozzle through and then tensioned around the nozzle, pressing the interface against the nozzle to form a seal, forming a bubble at the tip of the nozzle. After a small opening 140 is made in the cranium, dura and arachnoid mater tissue layers, an adjustable surface brace 112 is set to the appropriate length so that, when the injector 100 is braced against the outside of the head (for example, if brain interfacing is desired), the tip of the interface-wrapped nozzle rests at the appropriate depth for inflation.

In FIG. 2, the pump has begun inflating the bubble of interface 150 over the tip of nozzle 115 and the rest of the interface begins getting pulled through the tensioning encasement as inflation continues past the tensioning encasement and more interface is needed to form the growing surface of the bubble. The bubble expands outwards to fill the space on its periphery, putting an ever-larger portion of the interface in contact with the surface of the brain. Although the interface shown in these figures is relatively small, it is possible in theory to develop an interface that is large enough to contact the surface of, and thus interface with, an entire hemisphere of the brain and perhaps interface significant neural volumes and achieve high accuracy, high precision, high bandwidth communication, interacting with a majority of the entire human cortex. At this extreme, it may be possible to install, what is effectively, a full-cortex brain-computer interface using two hemispheric brain-computer interfaces and three small burr holes in the cranium. Two temporal holes to inject the interface through and an optional third small burr hole to allow CSF to drain out of the cranial cavity as it's displaced by the inflating interface. This is in stark contrast to the conventional technique where to build a full brain-computer interface with electrodes, it would be necessary to remove the entire cranium of the patient receiving the interface, which is a far more risky, technical, and complicated surgical option with a much longer recovery time than one or two small openings in the cranium.

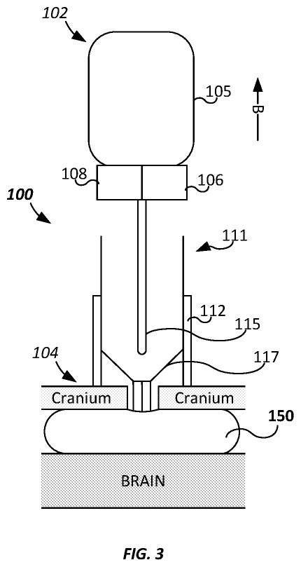

FIG. 3 shows the interface being fully inflated within the subarachnoid space, with the two units to allow the tensioning encasement 110 to pinch the opening of the interface shut and seal the bubble permanently. It is possible that this could be done by incorporating heating elements into the part of the tensioning encasement holding the interface under pressure. This sealing and a slight elasticity to the interface may ensure that the inflated membrane remains under enough pressure to push against the surface of the brain with enough force to ensure good contact. Proximity to the tissue being measured and stimulated is necessary for highest-quality data and interfacing, so it is important that the tensioning encasement produce a solid seal satisfactorily when the nozzle is withdrawn. It is also possible that most proximal portion of tensioning encasement clamp may function as an independent sealer, which pinches and seals the most proximal portion of interface within tensioning encasement 110 if nozzle is removed sufficiently such that tension clamp still seals bubble but complete pinching and sealing may be performed using most proximal portion of tension clamp and tensioning encasement arrangement from which nozzle has been removed. After full inflation and sealing, the two units may be withdrawn from the surgical portal, the section(s) of cranium removed for the procedure may be put back into place using an adhesive or surgical portal which accesses APT is otherwise closed surgically and the incision(s) in the scalp is sutured shut. Patients can then read the activity of the entire region of APT area interface contacts (at least to a specific accuracy up to a specific tissue depth given constant arbitrary accuracy threshold) and modulate signals within this region as well by the precise stimulation of tissue.

In some examples, tensioning encasement 110 may ensure that pumped fluid cannot escape the bubble of inflation and the biocompatible lubricant may ensure that the inflating interface slides through the tensioning encasement and off the nozzle with minimal resistance. In this way, the inflating interface easily slides off the nozzle and becomes incorporated into the ever-growing inflation bubble expanding over the surface of the APT. Inflation of this bubble stops slightly before the entire interface has slid through the tensioning encasement and the bubble is permanently sealed. In the case of the brain, cerebrospinal fluid (CSF) displaced by inflating the interface bubble may be able to escape around the inflating membrane or it could be drained out of a secondary small surgical portal, if necessary.

The sealed interface may be under enough pressure to firmly press against the surface of the target APT to ensure the best possible interface. After this, the section of the cranium removed for the purpose of inserting the interface can be replaced, the scalp can be sutured and the interface can begin reading brain activity and stimulating target regions of APT as desired. Optionally, injector 100 may have electronic control over tension or a mechanical spring clamp to achieve same desired tensioning parameters. The tensioning encasement may serve as a shield as well, functioning as a barrier to prevent the bubble from inflating out of the burr hole and forcing it to inflate into the surrounding subarachnoid space. The nozzle 115 of the pump system can be made as long as desired to facilitate the insertion of interface 100 into deeper bodily recesses, or to facilitate the insertion of larger interfaces by allowing more space for the un-inserted portion of the interface distal to the tension clamp and continuous with interface portion which constitutes inflation bubble to be pulled up past tension clamp and ring nozzle shaft like loose sock pulled over narrow shaft until extended entirely and then allowed to drape before being pinched against the shaft by the elastic band (tensioning encasement) at the proximal tip.

Surgeons installing interfaces generally will place the nozzle tip in contact with the part of the brain computer interface which is to rest directly below the surgical portal as defined by that point which is desired resting immediately below surgical portal when interface (at least if cavernous) is completely inflated within bodily confines as installed via designated surgical portal most probably. Assuming interface is properly placed on nozzle. It may be possible for the whole operation to be done robotically. In addition, it is possible to utilize a form of directable, laparoscopic nozzle capable of snaking its way into crevices and navigating tight spaces as insertion means. Finally, the tensioning encasement may be used as a means of sealing the opening of the interface after it has been fully inflated assuming it can pinch off the section of interface passing through it if nozzle is removed.

In some embodiments, the free tail 152 of the interface (the portion furthest away from the nozzle tip when loaded onto an injector) may be sealed using an ultraviolet (UV) sensitive adhesive coating on its inner surface. Optionally, free tail 152 may be twisted around via rotation of the tensioning encasement to pinch off the fluid interior of the interface from the outside and then sealed via exposure to UV light. In addition to being sealable using a UV sensitive adhesive, the neck of the interface may be sealed using a heated clamp (assuming the polymers used in its construction are amenable to heat-sealing). A built-in UV light or heat clamp can be integrated into the injector assembly or simply used as an additional tool during the implantation operation.

In some embodiments, a similar process may utilize two pumps in concert, one to pump fluid into the interface to inflate it at one location (inflator), and another to pull cerebrospinal fluid (CSF) from the subarachnoid space in amounts equal to the volume of fluid being pumped into the interface (sucker) at another. The two systems may contain sensors to gauge the amount of pressure being created, with electronic systems working in concert to ensure the suction pressure at the outlet is equal to the inflation pressure at the inlet and also making sure that the intracranial pressure does not deviate meaningfully from its natural setpoint. In this way, both pumps may work in concert at a wide range of combined, opposing pressures to coax an interface to unfurl over a large section of the brain or other APT. With the pressure of the fluid being pumped being substantially equal and opposite to the pressure of the fluid being removed, the risk of damage to any blood vessels due to the maintenance of a constant, net, intracranial, CSF pressure (with respect to blood pressure) may be reduced. By having the electronic systems work to maintain a net-zero intracranial pressure during inflation, the risk of bursting or collapsing blood vessels during the operation may be minimized.

One possible advantage of this method is that two small holes drilled in the skull (one to insert the interface and another smaller one to pump CSF out of the subarachnoid space) could be enough to install an interface over an entire hemisphere of the brain. The total area required for surgical access for the implantation would be dramatically smaller than the total area of brain that would be covered by an interface implanted using those holes. In some example, three small holes may be enough to interface both hemispheres. The need to fit the interface through at least one of the holes drilled is the only factor determining the invasiveness of the surgical operation for implantation. A small portal for the addition of a sealed suction nozzle may require a hole smaller in size than that required for an interface, and only a maximum of one such hole may be desired.

There is another possible advantage of this disclosed interface over traditional electrode-based brain-computer interfaces. Specifically, current art for electrode-based BCIs requires a removal of a section of skull that is nearly equal in area to the region of the brain being interfaced. Therefore, interfacing an entire hemisphere of the brain using electrode-based BCIs would require surgical resection of an entire hemisphere of scalp and skull. This is far more surgically intense and risky than simply drilling two small holes in the skull. Being its own self-contained BCI, all that is needed to have a working BCI is to implant an interface that sits over a surface of the region of the brain or other APT. The electrodeless configuration precludes the need to avoid any blood vessels (as there is no puncturing of brain tissue) as well, thus reducing surgical risks of implantation even further. In addition to the reduced surgical intensity of installation (per unit area of brain interfaced), the minimally-invasive nature of the implantation means reduced, post-operative recovery times and less scarring than an electrode-based BCI covering an equivalent area.

In some embodiments, the main structure allowing for insertion of an interface into subarachnoid space includes one or more push-pull chain segments 180 arranged around the central nozzle 115, which are redirected radially away from the nozzle 180 at the base of the nozzle so that downward pressure being applied to the push-pull chain segments causes directed, outward radial pressure against the bubble of interface formed below the tension ring. The radial arms 182 formed using the insertion tool push the interface into and through the subarachnoid space peripheral to skull hole drilled to insert. Strung between each radial arm 182 and its neighbors are thin strings of elastic material or filaments 184 which are fastened to the internal wall of the push-pull chain within the hollow cavity formed by chaining many hollow push-pull chain links 185a,185b together in series. In some examples, each radial arm 182 is coupled to two elastic filaments 184, each of which is attached at one end to the same radial arm, and which form unique attachments to either one or another neighboring radial arms (FIG. 6).

In FIG. 4, one arrangement of radial arms is shown with their guides stripped away except at the point where the radial arms are made to turn 90 degrees to be coplanar with the subarachnoid space. The filaments 184 may be seen strung between the termini of each radial arm, forming a perimeter that presses cavernous interfaces out laterally in all directions. The radial arms are arranged around a central nozzle, which, while not primarily how the interface is pushed laterally in this case, still may provide fluid pressure to the cavernous interface as it inflates. It should be noted that interface injectors do not have to include a central nozzle. For example, lateral expansion and fluid inflation may be performed separately. Downward pressure on the push pull chains may be translated into lateral expansion of radial arms in all directions. Some injectors may be able to control the direction in which the radial arm is projected. A containment ring 160 circles the interface, but that is also not necessary in some cases. In some cases, its function can be provided by the skull hole the interface is being inserted through. Below, an illustration of a possible arrangement whereby excess radial arm is deployed using electric motors. These windings provide space to retract the radial arms as well. These motors can double as force sensors to monitor the process of insertion for dangerous resistance and can be individually controlled by a computer or surgeon as well.

Various configurations of radial arms can be achieved by altering the number of push-pull chain length segments and dynamically controlling the radial extension of each radial arm 182 individually. The resulting configuration forms what is an expanding polygon, which has a perimeter bounded and formed by filaments 184. It will be understood that polygonal shapes may include three-sided, four-sided, five-sided, six-sided, seven-sided, eight-sided, nine-sides or ten-sided polygons defined by an equal number of arms 182, though theoretical polygons may have any number of distinct vertices that are greater than or equal to three. In some examples, the radial position of the vertices away from the nozzle depends upon how much length the push-pull chain segment responsible for creating that radial arm has been extended. Pumping fluids into interface may only be necessary occasionally if the push-pull chains and filaments are capable of generating sufficient expansion force required to push the interface a certain distance into subarachnoid space and to overcome the resistance of the interface being pulled through the tension ring as well as unfolding within bodily cavity in which it is implanted. In some examples, additional instrument parameters include achieving precise dynamic control of the expansion using digital control systems and/or surgical supervision. Push-pull chain segments may be oriented and driven over tracks 187 which guide radial arm formation by redirecting downward motion outward. This can be achieved using specialized tracks, or simply by containing and orienting a push-pull chain segment in an enclosed environment of specific geometric cross section. Wrapped interfaces may fit around the tracks for the radial arms so that the radial arms would extend outward into subarachnoid space within interface confines after cavernous interface is placed over nozzle and/or radial arm push pull chain guide structure which positions both pumped fluid and radial arms below skull level and in subarachnoid space.

In some examples, a hollow cylinder with guides for specialized radial arms with a key protruding off the side of the hollow cylinder which connects to the side of the push pull chain which is capable of bending more holds a folded interface which is attached to the radial arms and then retracted so that it fits within the hollow cavity formed within hollow cylinder. This cylinder is placed in the skull hole and the radial arms are deployed, with a flange at the outlet redirecting them 90 degrees so they are coplanar with the subarachnoid space immediately adjacent to insertion region. The same control mechanisms as are present in the interface injector previously described can be used to control the deployment of the various radial arms which are attached at various points to the interface so that it can be expanded within the subarachnoid space.

Interface injectors may combine any number of elements and/or mechanisms described herein, and may be forced laterally within subarachnoid space so they may interface with larger APT surface areas and volumes than those through which they are inserted. This may include, exclusively radial arms, or combined radial arms and/or fluid pump and/or fluid pump(s) alone. Combinations of these basic design principles may provide a versatile platform for inserting manifold interfaces of differing dimensions, geometries, topological configurations into a variety of anatomical domains where APT is present. Two main types detailed being solid, cylindrical nozzle interface injectors for the insertion of balloon-shaped, cavernous injectors and hollow, cylindrical nozzle injectors for the insertion of flat, non-balloon-shaped, non-cavernous interfaces.

In some examples, radial arms may provide the majority of lateral outward pressure causing interface expansion within the subarachnoid space, whereas fluid pumping takes on a secondary role filling the volume of the expanding interface, pressing it against cranial meninges and/or APT surface. This is because, fluid pressure inside the interface may increase in proportion to surface area, and even small pressures within interface when distributed across large enough APT areas may generate significant net force compressing APT tissue capable of damaging or causing harm as expansion increases beyond certain size limit, which may cause tissue damage. It is therefore likely that, in some embodiments, radial arms will perform primary lateral expansion role in any given interface injector, while fluid pumping takes on a secondary role ensuring cavernous interface is inflated post-expansion such that it is braced against meningeal cranial surface and/or brain (for example when implanted in skull). This ensures implanted interfaces remain fixed in position.

One example of a non-cavernous interface is shown in FIGS. 7A-D, where the interfaces 250 is shown as a generally flat, non-balloon surface that is pressed into place with injector 200 via radial arms. In this examples, one or more terminal ends of the interface 250 may be coupled, linked or mated with the radial arms. For examples, the radial arms may be coupled at their distal end to pockets 251 that accept clips 251 that are coupled to the radial arms. In some examples, the clips 251 are linked or coupled to the pockets during insertion and expansion of interface 250, but are then released form the pocket after insertion so that the radial arms can be retrieved, leaving the interface 250 in position. It will be understood that two, three, four or more arms are possible as previously described, and that additional insertion or adjustment aids (e.g., fluid pumps and the like) may be used in addition to this basic hollow injector.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

1. A computer-brain interface injector, comprising:
a hollow tube having a longitudinal axis and configured to receive at least a portion of an interface; and
at least two arms capable or radial extension in a direction normal to the longitudinal axis of the hollow tube.

2. The computer-brain interface injector of claim 1, wherein the at least two arms comprise at least three arms.

3. The computer-brain interface injector of claim 1, wherein the at least two arms comprise six arms.

4. The computer-brain interface injector of claim 1, further comprising filaments that extend between each of the at least two arms.

5. The computer-brain interface injector of claim 1, further comprising:
a first unit including a fluid reservoir, a pump coupled to a power source, and a nozzle in fluid communication with the fluid reservoir; and
a second unit including a injector support structure, the second unit being coupleable to the first unit.

6. The computer-brain interface injector of claim 5, wherein the at least two arms are disposed farther radially than the nozzle, which is centrally located.

7. The computer-brain interface injector of claim 5, wherein the nozzle extends from the fluid reservoir past the second unit.

8. The computer-brain interface injector of claim 5, wherein the second unit comprises a surface brace.

9. The computer-brain interface injector of claim 5, wherein the second unit comprises a tapered guiding surface.

10. A system comprising:
the computer-brain interface injector of claim 5; and
a magnetic brain-computer interface.

11. The system of claim 10, wherein the magnetic brain-computer interface is balloon-shaped and includes an opening that partially receives a portion of the nozzle.

12. The system of claim 11, further comprising a sealing element to close the opening of the balloon-shaped magnetic brain-computer interface.

13. The system of claim 11, wherein the sealing element comprises UV light.

14. The system of claim 11, further comprising an containment ring disposed about a section of the balloon-shaped magnetic brain-computer interface.

15. A method of delivering a computer-brain interface, comprising:
providing a hollow tube having a longitudinal axis and configured to receive at least a portion of an interface, and at least two arms capable or radial extension in a direction normal to the longitudinal axis of the hollow tube; and
guiding a magnetic brain-computer interface with the at least two arms.

16. The method of claim 15, further comprising providing a first unit including a fluid reservoir, a pump coupled to a power source, and a nozzle in fluid communication with the fluid reservoir, and a second unit including an injector support structure, tensioning encasement and interface subarachnoid expansion mechanisms, the second unit being coupleable to the first unit, and inflating a balloon-shaped magnetic brain-computer interface with fluid from the fluid reservoir via the nozzle.

17. The method of claim 15, further comprising coupling the least two arms with filaments.

18. The method of claim 16, further comprising delivering the balloon-shaped magnetic brain-computer interface in a subarachnoid space between a brain and a cranium.

19. The method of claim 18, further comprising expanding the balloon-shaped magnetic brain-computer interface radially outward after a portion of the balloon-shaped magnetic brain-computer interface has been delivered below the cranium.

20. The method of claim 16, further comprising forming a burr hole and extending the nozzle at least partially through the burr hole.
